(19) United States
(12) Patent Application Publication
(10) Pub. No.: US 2009/0029900 A1
(43) Pub. Date: Jan. 29, 2009

(54) Personal care article for sequentially dispensing compositions with distinct fragrance characters

(75) Inventors: Jonathan Robert Cetti, Mason, OH (US); Christopher Dean Putman, West Chester, OH (US); Andres Ernesto Velarde, Cincinnati, OH (US); Karen Lehnhoff, Cincinnati, OH (US); Edward Dewey Smith, III, Mason, OH (US); Julien Claude Plos, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company

(21) Appl. No.: 11/881,535

(22) Filed: Jul. 27, 2007

(51) Int. Cl. B65D 88/54; A61K 8/18

(52) U.S. Cl. 512/1; 222/319

(57) ABSTRACT

The present invention relates to a personal care article that comprises a single chamber package and a liquid personal care product. The package comprises a dispensing orifice, a first zone proximate to the dispensing orifice and a second zone distal to the dispensing orifice. The liquid personal care product comprises a first personal care composition substantially disposed within the first zone and the second personal care composition substantially disposed within the second zone. The first composition comprises a first fragrance character and the second composition comprises a second fragrance character; wherein the first fragrance character is distinct from the second fragrance character.

### FIELD OF THE INVENTION

The present invention relates to a personal care article that provides a liquid personal care product that comprises at least two compositions each having a fragrance character which is distinct from each other.

### BACKGROUND OF THE INVENTION

Personal care compositions are well known and widely used for cleansing and moisturizing skin and hair, delivering actives, hiding imperfections, to reducing the oiliness/shine, as well as, providing scent to the shower and/or the skin. The efficacy of these types of compositions is directly related to the frequency of use. Consumers often habituate or tire of a particular scent of a personal care composition over time. When this habituation occurs consumers often decrease or even or stop use of one personal care product and begin to use another personal care product of with another scent despite the benefits gained by compliant use of the first personal care product over time. With the space in the shower or bath being limited, a typical shower or bath does not have enough space for multiple containers of personal care compositions having different fragrances, so that consumers can easily switch between them.

### SUMMARY OF THE INVENTION

The present invention relates to a personal care article that comprises a single chamber package and a liquid personal care product. The package comprises a dispensing orifice, a first zone proximate to the dispensing orifice and a second zone distal to the dispensing orifice. The liquid personal care product comprises a first personal care composition substantially disposed within the first zone and the second personal care composition substantially disposed within the second zone. The first composition comprises a fragrance formulation having a first fragrance character and the second composition comprises a second fragrance formulation having a second fragrance character; wherein the first fragrance character is distinct from the second fragrance character. Thus, the dispensed liquid personal care product changes in fragrance character over the product lifetime, which overcomes the problem of consumer habituation to a scent.

### BRIEF DESCRIPTION OF THE DRAWINGS

FIGS. 1A and 1B illustrate a personal care article with three zones having horizontal interfaces between the compositions in each zone.

FIGS. 2A and 2B illustrate a personal care article with two zones having diagonal interfaces between the compositions and the zones

FIGS. 3A and 3B illustrate a personal care article with two zones having horizontal interfaces between the compositions and the zones.

FIG. 4 illustrates a diagram of the tubing used in the migration testing of the partitioned perfume components.

FIGS. 5A and 5B illustrate a personal care article wherein the package is in the form of a tottle.

FIGS. 6A, 6B and 6C illustrate a personal care article wherein the package is in the form of a bottle.

FIG. 7 depicts a chart rating consumer's anticipation of a change in fragrance over the time of usage.

### DETAILED DESCRIPTION OF THE INVENTION

The term “ambient conditions” as used herein, refers to surrounding conditions at one (1) atmosphere of pressure, 50% relative humidity, and 25° C.

As used herein, “comprising” means that other steps and other ingredients which do not affect the end result can be added. This term encompasses the terms “consisting of” and “consisting essentially of”. The compositions and methods/processes of the present invention can comprise, consist of, or consist essentially of the essential elements and limitations of the invention described herein, as well as any of the additional or optional ingredients, components, steps, or limitations described herein useful in personal cleansing compositions intended for topical application to the hair or skin.

The term “liquid” as used herein means that the composition is generally flowable to some degree. “Liquids”, therefore, may include liquid, semi-liquid, cream, lotion or gel compositions intended for topical application to skin. The compositions may exhibit a viscosity of equal to or greater than about 1,500 (centipoise, hereinafter “cps”), equal to or greater than about 3,000 cps, equal to or greater than about 5,000 cps, equal to or greater than about 10,000 cps or equal to or greater than about 20,000 cps and no more than about 1,000,000 cps, no more than about 500,000 cps, no more than about 300,000 cps, or no more than about 200,000 cps as measured by the T-Bar Viscosity Method described hereinafter.

The term “package” includes any suitable container for a personal care compositions exhibiting a viscosity from about 1,500 centipoise (cP) to about 1,000,000 cP, of including but not limited to bottle, tottle, tube, jar, aerosol container, pressurized containers, non-aerosol pump and mixtures thereof.

The term “personal care composition” as used herein, refers to compositions intended for topical application to the skin or hair. The compositions of the present invention are rinse-off formulations, in which the product is applied topically to the skin or hair and then is subsequently rinsed within minutes from the skin or hair with water, or otherwise wiped off using a substrate with deposition of a portion of the composition. The compositions also may be used as shaving aids. The personal care composition of the present invention is typically extrudable or dispensible from a single chamber package. The personal care compositions of the present invention can be in the form of liquid, semi-liquid, cream, lotion or gel compositions intended for topical application to skin. Examples of personal care compositions of the present invention can include but are not limited to shampoo, conditioning shampoo, hair conditioner, body wash, moisturizing body wash, shower gels, skin cleansers, cleansing milks, hair and body wash, in shower body moisturizer, pet shampoo, shaving preparations and cleansing compositions used in conjunction with or applied to a disposable cleansing cloth. The personal care compositions of the present invention are typically in the form of a liquid. The product forms contemplated for purposes of defining the compositions and methods of the present invention are rinse-off formulations by which it is meant that the product is applied topically to the skin or hair and then subsequently (i.e., within minutes) rinsed away with water, or otherwise wiped off using a substrate or other suitable removal means.

The term “fragrance formulation” or “perfume formulation” as used herein, refers to formulations intended for providing a fragrance character to a personal care composition. The fragrance formulations or perfume formulations of the present invention are mixtures of multiple partitioned perfume components.

The term “stable” as used herein, unless otherwise specified, means that the compositions of the personal care product maintain at least two “separate” compositions when sitting in physical contact at ambient conditions for a period of at least I week according to the dialysis method described hereinafter. By “separate”, it is meant that there is substantially no mixing of at least one partitioned perfume component of two compositions proximate to each other with the personal care article, such that less than 30% of the concentration of at least one partitioned perfume component of interest within the first composition migrates to the second composition proximate to first composition or said first composition maintains a distinct fragrance character from the fragrance character of said second composition according to the fragrance differentiation method described hereinafter. The partitioned components of interest are detected by the Gas Chromatograph method described hereinafter. For example that is not considered “stable” as defined is the partitioned component Fructone, which has a C log P of 0.68. Using the dialysis method, analytical measurements indicate that 34% of the Fructone concentration had migrated from a composition containing Fructone into the opposite side of the dialysis cell, a composition not containing Fructone.

The term “structured,” as used herein means having a rheology that confers stability on the personal care composition. The degree of structure is determined by characteristics determined by one or more of the following methods the Yield Stress Method, or the Zero Shear Viscosity Method or by the Ultracentrifugation Method, all in the Test Methods below. Accordingly, a surfactant phase of the composition of the present invention is considered “structured,” if the surfactant phase has one or more of the following properties described below according to the Yield Stress Method or the Zero Shear Viscosity Method or by the Ultracentrifugation Method. A surfactant phase is considered to be structured, if the phase has one or more of the following characteristics:
A. a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter:
B. a Zero Shear Viscosity of at least about 500 Pascal-seconds (Pa-s), preferably at least about 1,000 Pa-s, more preferably at least about 1,500 Pa-s, even more preferably at least about 2,000 Pa-s; or
C. a Structured Domain Volume Ratio as measured by the Ultracentrifugation Method described hereafter, of greater than about 40%, preferably at least about 45%, more preferably at least about 50%, more preferably at least about 55%, more preferably at least about 60%, more preferably at least about 65%, more preferably at least about 70%, more preferably at least about 75%, more preferably at least about 80%, even more preferably at least about 85%.

The term “surfactant component” as used herein means the total of all anionic, nonionic, amphoteric, zwitterionic and cationic surfactants in a phase. When calculations are based on the surfactant component, water and electrolyte are excluded from the calculations involving the surfactant component, since surfactants as manufactured typically are diluted and neutralized.

As used herein “tottle” refers to a bottle which rests on neck or mouth which its contents are filled in and dispensed from, but it is also the end upon which the bottle is intended to rest or sit upon (e.g., the bottle's base) for storage by the consumer and/or for display on the store shelf (this bottle is referred to herein as a “tottle”). Typically, the closure on a tottle is flat or concave, such that the tottle, when stored, rests on the closure. Suitable tattles are described in the co-pending U.S. patent application Ser. No, 11/067443 filed on Feb. 25, 2005 to McCall, et al, entitled “Multi-phase Personal Care Compositions, Process for Making and Providing, and Article of Commerce.”

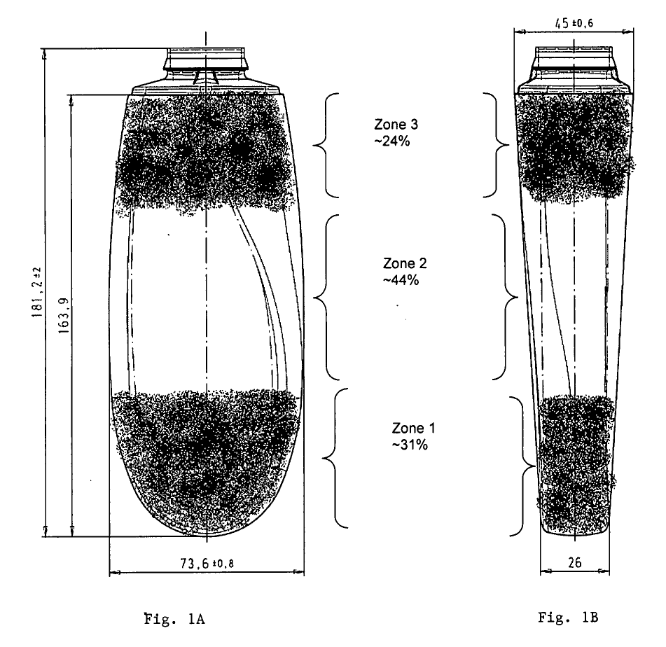

As used herein the term “zone” is a domain or region within a single chamber package which corresponds to a composition of the personal care product. An interface between the zones can be distinct or gradual or separated by another zone. The amount contained in a zone can be defined by a percentage of the package volume and a zone comprises at least 10% of the package volume of a given package as shown in FIGS. 1A, 1B, 2A, 2B, 3A and 3B of the present invention.

All percentages, parts and ratios are based upon the total weight of the compositions of the present invention, unless otherwise specified. All such weights as they pertain to listed ingredients are based on the active level and, therefore; do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about.” All molecular weights as used herein are weight average molecular weights expressed as grams/mole, unless otherwise specified.

The present invention relates to a personal care article that provides a single chamber package that comprises a liquid personal care product. The liquid personal care product comprises at least two personal care compositions, each composition having a noticeably distinct fragrance character as defined by the fragrance differentiation method described hereinafter. The respective fragrances of the personal care compositions remain distinct although the compositions are contained within the package. These distinct fragrances are dispensed sequentially from the package. For example, a package could dispense a vanilla scented personal care composition, followed by a lavender scented composition, followed by a vanilla scented personal care composition. Thus, the liquid personal care product changes in fragrance as it is dispensed from the package which overcomes the problem of consumer habituation to scent.

It is known in the art that multiple compositions can be held separate such as is disclosed in U.S. Pat. No. 6,787,511 to Patel (hereinafter referred to as the '511 patent), for example, which two aqueous compositions are contained within a single chamber package, wherein, when standing, the aqueous compositions form two or more visibly distinct aqueous compositions and, when agitated, the composition forms a visible single composition product.

In contrast to the present invention, the product described in the '511 patent is intended to be shaken to deliver the intended benefit. The viscosities of the individual compositions are disclosed in the '511 patent are such that the viscosity of the mixture is greater than the viscosity of either of the layers alone. The viscosities of the two compositions of the '511 patent art are represented by LYNX® Speed Shower Shake (containing maltodextrin, sodium chloride, surfactant, water and minors). The viscosities of the two compositions by LYNX® Speed Shower Shake were measured and found to be 26 centipoise for the lower composition and 1,203 centipoise for the upper composition, which are significantly lower than the disclosed viscosities of the compositions described in the subject invention. Thus, agitation of the product described in the '511 patent is needed to deliver the viscosity appropriate for the intended use. Furthermore, the fragrance of the lower composition and fragrance of the upper composition of the product described in the '511 patent are not distinct according the fragrance differentiation method.

The present invention relates to a personal care article for providing at least two liquid personal care compositions. The personal care article comprises a single chamber package and a liquid personal care product. The package comprises a dispensing orifice, a first zone proximate to the dispensing orifice and a second zone distal to the dispensing orifice. The liquid personal care product comprises a first personal care composition substantially disposed within the first zone and the second personal care composition substantially disposed within the second zone. The first personal care composition is substantially dispensed prior to dispensing the second composition, so that there is a limited amount of mixing of the fragrances of the first composition with that of the second composition. The term “substantially dispensed” as used herein, unless otherwise specified, means that at least 10%, or at least 25%, or at least 50% of said first personal care composition substantially disposed within said first zone is dispensed prior to the dispensing of the second personal care composition substantially disposed within the second zone. In one aspect, the first zone is in physical contact with the second zone within the single chamber package. In one aspect, the first personal care composition is in physical contact with the second personal care composition within the single chamber package. In one aspect, the personal care article is not intended to be shaken such that the first personal care composition mixes with the second personal care composition prior to dispensing the personal care compositions within the single chamber package.

The personal care article for dispensing and or applying at least two liquid personal care compositions that comprises a single chamber package that comprises at least two zones with at least two personal care compositions substantially disposed within the respective zones. The number of zones with a package and thus, the number of personal care compositions disposed within the respective zone can vary in number. For example, the package may have three zones and three personal care composition within the respective zones; four zones and four compositions, five zones and five compositions, and so on. In one aspect, the personal care article comprises a third zone medial to the dispensing orifice. In one aspect, the personal care article comprising a third personal care composition substantially disposed within the third zone; the third personal care composition comprising a third fragrance character is distinct from the first fragrance character and the second fragrance character concentration. In another aspect, the first zone, the second zone and the third zone comprise an equal percentage, by volume, of the package.

In another aspect, each personal care composition may comprise a dye, colorant or the like, such that each personal care composition is a distinct color or hue. For example, the first personal care composition can be a yellow color, the second personal care composition can be an orange color and the third personal care composition can be a purple color.

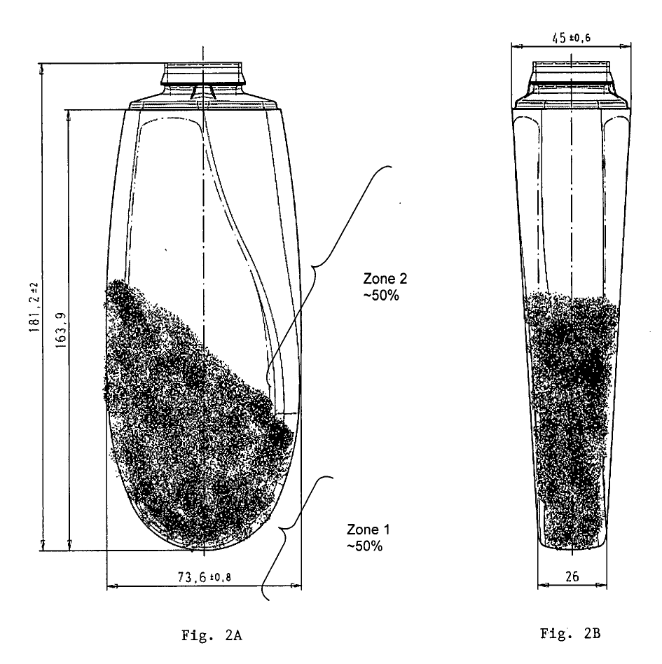

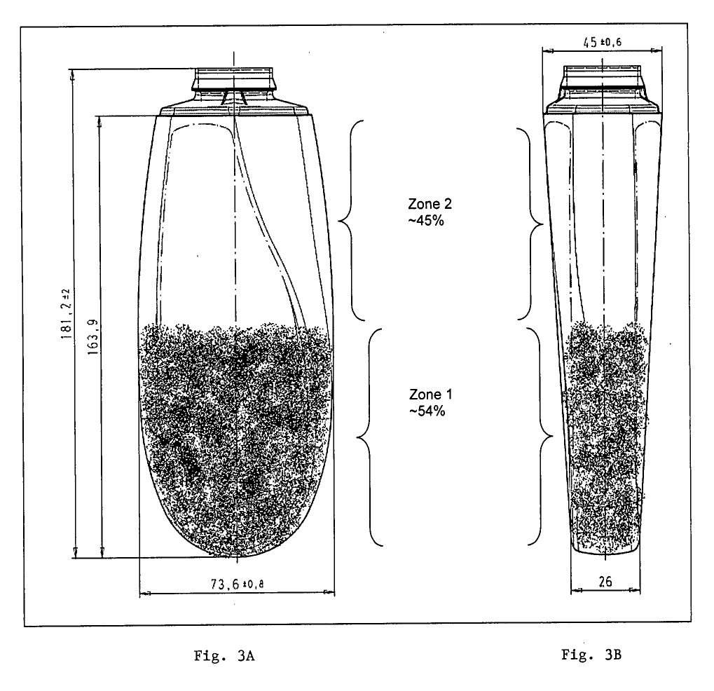

FIGS. 1A and 1B illustrate a personal care article with three zones with horizontal interfaces between the zones. As shown in FIGS. 1A and 1B, zone 1 is approximately 31% of the package volume, zone 2 is approximately 44% of the package volume and zone 3 is approximately 24% of the package volume. FIGS. 2A and 2B illustrate a personal care article with two zones having diagonal interfaces between the compositions and the zones. As shown in FIGS. 2A and 2B, zone 1 and 2 are approximately 50% of the package volume. FIGS. 3A and 3B illustrate a personal care article with two zones having horizontal interfaces between the compositions and the zones. As shown in FIGS. 3A and 3B, zone 1 is approximately 54% of the package volume and zone 2 is approximately 45% of the package volume.

The personal care compositions of the present invention comprise “partitioned perfume components” or “partitioned components”. The partitioned perfume components or partitioned components of the present invention are those small molecules which are capable of being partitioned into two or more separate compositions. The term “small molecules” refers to any material that has a molecular weight less than 1000 and is capable of being maintained or dispersed in a surfactant containing phase.

Not being bound by theory, the inventors believe that stability of a personal care composition can be enhanced if one chooses to use partitioned perfume components in personal care composition that have a higher C log P and are more hydrophobic and to avoid partitioned perfume components that have a lower C log P and are more hydrophilic. Preferably, the C log P of the partitioned perfume component is at least 2.

Furthermore, not to be bound by theory, the inventors believe that the stability of a personal care composition can be further enhanced if one chooses to use partitioned perfume components in personal care compositions that have a smaller molar volume and are more stable when dispersed or maintained in the surfactant phase and to avoid partitioned perfume components that have a higher molar volume and are less stable when dispersed or maintained in the surfactant phase. The molar volume as determined hereinafter is at least from about 50, or at least from about 75, or at least from about 100 cm³/mol to about 200, or to about 300, or to about 400 cm³/mol.

Even furthermore, not to be bound by theory, the inventors believe that the stability of a personal care composition can be further enhanced if one chooses to use personal care compositions with higher zero-shear viscosities and to avoid personal care compositions with lower zero-shear viscosities. Preferably, the zero-shear viscosity is at least 500 Pascal-s, or at least 1000 Pascal-s, or at least 1500 Pascal-s.

The personal cleansing product comprises at least one partitioned perfume component in the first composition that is not contained in the second composition of the personal care product. A composition may comprise from about 0.00001%, from about 0.001%, or from about 0.005% to about 10%, to about 2%, to about 0.1%, or to about 0.05%, by weight of the composition of a partitioned perfume component. In one aspect of the personal care article of the present invention, the first personal care composition or the second composition of the present invention may comprise a concentration of 0% partitioned perfume agents.

To enhance the benefit of the present invention, it is important that the partitioned components incorporated remain stable and do not migrate from one phase to the other. The Partition Coefficient Values (c Log P) reflect a molecule's hydrophilicity and thus the c Log P calculations are considered for the present invention to determine if they are appropriate to resist migration within the particular zones of the present invention. It has been found that partitioned components with a c Log P greater than 2 will resist migration in liquid personal care compositions. In one aspect, the first personal care composition comprises a first partitioned perfume component having a c Log P of at least 2 and the second personal care composition comprises a second partitioned perfume component having a c Log P of least 2.

c Log P and molar volume can be calculated for a variety of partitioned components with relatively good agreement between the protocols used to calculate them. According to the present invention, the protocol from ACD Labs website was used (www.acdlabs.com). In cases where the partitioned component contains ionizable groups, cLogD (variation of c Log P with pH) is used at the relevant composition pH. C log P is a calculated quantity for a partitioned component, determined by a mathematical algorithm using molecular substructure or fragment contributions with correction factors. The approach is common in such fields as toxicology, environmental transport, and pharmaceuticals, for example to facilitate development of drugs, especially for topical drugs that interact with lipid bilayers in skin, a molecular mechanism not dissimilar to interaction of partitioned perfume components with surfactant. Different substructure fragment algorithms exist which can calculate different C log P values for the same molecule, based on differences in algorithms and/or coefficients, as can be found in scientific literature. For the purposes of our invention, C log P is determined using the algorithm from Advanced Chemistry Development Labs as referenced and updated in the scientific literature (Hansch, C. and Leo, A., Substituent Constants for Correlation Analysis in Chemistry and Biology, Wiley Interscience New York (1979); updated in Leo., A. and Hoekman, D., Perspect. in Drug Discov. & Design, 18, 19 (2000)), whereas the value of Molar Volume and C log P were obtained using the ACD/I-lab web service (ACD/Molar Volume 8.02 and ACD/log P 8.02)

Accordingly, the partitioned components of the present invention may have a c Log P value of at least about 2, at least about 3, at least about 4, or at least about 5. Certain partitioned components, however, are effectively insoluble in either phase thus making it difficult to calculate a c Log P value, which essentially do not migrate, therefore are stable in the zones within the personal care product.

TABLE 1
Examples of Partitioned Perfume Components in the form of
Perfume Raw Materials with ClogP Values greater than 2.0

| Chemical Name | Clog P |
|---|---|
| Florol | 2.00 |
| Isoeugenol | 2.45 |
| Cis Jasmone | 2.905 |
| Dihydromyrcenol | 3.004 |
| Citral | 3.169 |
| Geraniol | 3.279 |
| Linalool | 3.281 |
| Citronellol | 3.382 |
| Cymal | 3.722 |

While not being limited to the following, the inventors have included the following experimental examples to illustrate properties of partitioned perfume components.

The migration of perfume components were measured in Composition A and Composition B. Composition A and Composition B in Table 2 are made with different fragrance formulations from Table 3 each having different fragrance components. Composition A and Composition B were made by conventional mixing techniques in the order of addition indicated. Addition steps 7 and 8 are premixed prior to addition to the main batch.

TABLE 2
Composition A and Composition B

| | (%) of Ingredients in Composition | |
|---|---|---|
| Composition Ingredients | Composition A | Composition B |
| 1. Sodium Trideceth-3 Sufate | 8.5 | 8.5 |
| 2. Sodium Lauryl Sulfate | 8.5 | 8.5 |
| 3. Sodium Lauroamphoacetate | 5.0 | 5.0 |
| 4. Disodium EDTA | 0.15 | 0.15 |
| 5. Sodium Benzoate | 0.2 | 0.2 |
| 6. Citric Acid | 1 | 1 |
| 7. PEG-90M | 0.15 | 0.15 |
| Xanthan Gum | 0.22 | 0.22 |
| Tridecyl Alcohol | 2 | 2 |
| 8. Water | QS | QS |
| Hydroxypropyl Guar | 0.6 | 0.6 |
| Sodium Chloride | 4.75 | 4.75 |
| 9. Kathon C | 0.0005 | 0.0005 |
| 10. Fragrance formulation A | 1.0 | — |
| 10. Fragrance formulation B | — | 1.0 |

TABLE 3
Fragrance Formulations for Composition A and Composition B
Components of Fragrance Formulations

| Fragrance Formulation A | Fragrance Formulation B |
|---|---|
| Fructone | Methyl dihydrojasmonate |
| p-Hydroxy phenyl butanone | Phenoxy ethyl butyrate |
| Liffarome | Dihydromyrcenol |
| Me-Ph-Carbinyl acetate | d-Limonene |
| Beta ionone | Hexyl cinnamic aldehyde |
| Geranyl acetate | Galaxolide |

After the compositions were made, Composition A and Composition B were placed in a dialysis cell according to the dialysis method.

Compositions A and B were analyzed according to the Gas Chromatograph method. The migration of the fragrance components were analyzed in each of the compositions. Composition A was analyzed for the partitioned perfume components of fragrance formulation B. Composition B was analyzed for the partitioned perfume components of fragrance formulation A. The results of the Gas Chromatograph are shown in Table 4 and Table 5 below. Results showed that partitioned perfume components with low C log P components have a greater tendency to migrate than partitioned perfume components with a higher C log P.

TABLE 4
Percent migration of fragrance components from fragrance
formulation A as analyzed in Composition B

| Fragrance Formulation A | | | |
|---|---|---|---|
| Molar | | | |
| Volume | CLogP | PRM | % migration |
| 163.2 | 0.68 | Fructone | 34.0 |
| 150.8 | 0.93 | p-Hydroxy phenyl | 25.7 |
| | | butanone | |
| 164.0 | 2.48 | Liffarome | 16.9 |
| 175.6 | 2.65 | Me-Ph-Carbinyl acetate | 22.0 |
| 203.6 | 3.85 | Beta ionone | Not detected |
| 233.4 | 4.47 | Geranyl Acetate | Not detected |

TABLE 5
Percent migration of fragrance components from fragrance
formulation B as analyzed in Composition A

| | | | % migration of |
|---|---|---|---|
| Fragrance Formulation B | | | fragrance materials |
| Molar | | | from formulation B as |
| Volume | CLogP | PRM | analyzed in formulation A |
| 199.8 | 2.49 | Phenoxy ethyl butyrate | Not detected |
| 203.1 | 3.53 | Dihydromyrcenol | Not detected |
| 229.8 | 2.50 | Methyl dihydrojasmonate | Not detected |
| 163.2 | 4.45 | d-Limonene | Not detected |
| 226.5 | 5.33 | Hexyl cinnamic aldehyde | Not detected |
| 273.2 | 6.23 | Galaxolide | Not detected |

The inventor was able to conclude from this data that fragrance components Beta ionone, p-Hydroxy phenyl butanone, Liffarome, Me-Ph-Carbinyl acetate, Geranyl Acetate, Phenoxy ethyl butyrate, Dihydromycenol, Methyl dihydrojasmonate, d-Limonene, Hexyl cinnamic aldehyde, and Galaxolide could be considered stable partitioned perfume components. These partitioned perfume components are stable and can be used in a two fragrance product. Fructone would not be considered stable partitioned perfume component because more than 30% of this perfume component migrated from one composition to another.

The migration of fragrance components was tested in five compositions C, D, E, F and G. These compositions vary in level of surfactant, type of surfactant, addition of structurant and end product viscosity. The compositions which are made with fragrance formulation C from Table 9 are designated as 1 (e.g. C₁, D₁, E₁, F₁ and G₁). The same compositions only without the addition of fragrance formulation C are designated by 2 (e.g. C₂, D₂, E₂, F₂ and G₂). The compositions in Tables 6, 7 and 8 were prepared according to conventional mixing techniques using the order of addition indicated. Addition step 8 in Table 6 and Table 7 containing Tridecyl Alcohol, PEG-90M, Xanthan Gum and Hydroxypropyl Guar was premixed prior to addition to the batch. The compositions in Table 8 was prepared according to conventional mixing techniques in the order of addition indicated. Addition step 6 in Table 8 containing water and Polyquaternium-10 was premixed prior to addition to the batch.

TABLE 6
Composition C₁, C₂, D₁ and D₂

| | C₁, | C₂ | D₁ | D₂ |
|---|---|---|---|---|
| 1. Water | QS | QS | QS | QS |
| 2. Sodium Trideceth-3 Sulfate | 8.5 | 8.5 | 8.5 | 8.5 |
| 3. Sodium Lauryl Sulfate | 8.5 | 8.5 | 8.5 | 8.5 |
| 4. Sodium Lauroamphoacetate | 5.0 | 5.0 | 5.0 | 5.0 |
| 5. Disodium EDTA | 0.15 | 0.15 | 0.15 | 0.15 |
| 6. Sodium Benzoate | 0.2 | 0.2 | 0.2 | 0.2 |
| 7. Sodium Chloride | 4.7 | 4.7 | 4.7 | 4.7 |
| 8. Tridecyl Alcohol | 2 | 2 | 2 | 2 |
| PEG-90M | 0.15 | 0.15 | 0.15 | 0.15 |
| Xanthan Gum | 0.22 | 0.22 | 0.22 | 0.22 |
| Hydroxypropyl Guar | 0.6 | 0.6 | — | — |
| 9. Citric Acid | 0.84 | 0.84 | 0.87 | 0.87 |
| 10. Fragrance Formulation C | 1 | | 1 | |
| 11. Methyl Chloro Isothiazolinone & Methyl Isothiazolinone | 0.0005 | 0.0005 | 0.0005 | 0.0005 |
| pH | 5.8 | — | 5.7 | — |
| Zero-shear Viscosity (Pa · s) | 2090 | — | 1277 | — |

TABLE 7
Composition E₁, E₂, F₁ and F₂

| | E₁ | E₂, | F₁ | F₂ |
|---|---|---|---|---|
| 1. Water | QS | QS | QS | QS |
| 2. Sodium Tridecth-3 Sulfate | 4.25 | 4.25 | 3.8 | 3.8 |
| 3. Sodium Lauryl Sulfate | 4.25 | 4.25 | 3.8 | 3.8 |
| 4. Sodium Lauroamphoacetate | 2.5 | 2.5 | 2.25 | 2.25 |
| 5. Disodium EDTA | 0.15 | 0.15 | 0.135 | 0.135 |
| 6. Sodium Benzoate | 0.2 | 0.2 | 0.18 | 0.18 |
| 7. Sodium Chloride | 2.4 | 2.4 | 2.2 | 2.2 |
| 8. Tridecyl Alcohol | 2 | 2 | 1.8 | 1.8 |
| Xanthan Gum | 0.22 | 0.22 | 0.2 | 0.2 |
| Hydroxypropyl Guar | 0.6 | 0.6 | 0.54 | 0.54 |
| 9. Citric Acid | 0.46 | 0.46 | 0.42 | 0.42 |
| 10. Petrolatum | — | — | 7 | 7 |
| 11. Mineral Oil | — | — | 3 | 3 |
| 12. Fragrance Formulation C | 1 | — | 1 | — |
| 13. Methyl Chloro Isothiazolinone & Methyl Isothiazolinone | 0.0005 | 0.0005 | 0.0005 | 0.0005 |
| pH | 5.7 | — | 5.7 | — |
| Zero-shear Viscosity (Pa · s) | 418 | — | 828 | — |

TABLE 8
Composition G₁ and G₂

| | G₁ | G₂ |
|---|---|---|
| 1. Sodium Lauryl Sulfate | 9.5 | 9.5 |
| 2. Sodium Laureth Sulfate | 5.7 | 5.7 |
| Adjust to pH 5 with Citric Acid | | |
| 3. Acrylates Copolymer (Aqua SF-1) | 1.9 | 1.9 |
| 4. Coco Monoethanolamide | 0.76 | 0.76 |
| 5. Caustic Soda 50% | 0.16 | 0.16 |
| 6. Water | QS | QS |
| Polyquaternium-10 | 0.24 | 0.24 |
| 7. Disodium EDTA | 0.12 | 0.12 |
| 8. Sodium Benzoate | 0.24 | 0.24 |
| 9. Citric Acid | 0.65 | 0.65 |
| 10. Fragrance Formulation C | 1 | — |
| 11. Methyl Chloro Isothiazolinone & Methyl | 0.0005 | 0.0005 |
| Isothiazolinone | | |
| pH | 5.4 | — |
| Zero-shear Viscosity (Pa · s) | 553 | — |

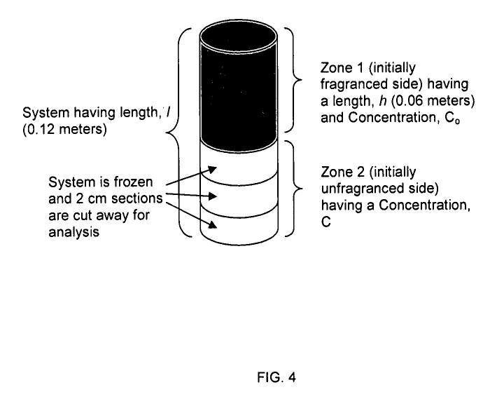

Compositions C, D, E, F, G and H in Tables 6, 7 and 8 were prepared and were filled in a 1″ diameter tubing (Inner Diameter 1 inch, Outer Diameter 1.25 inch, Wall ⅛ inch, supplied by Saint-Goban Performance Plastics). The tube diagram is shown in FIG. 4. Section 1 of each tube is the first 6 cm of the tubing. Section 1 contains the composition with the fragrance formulation C. In each case, section 1 has a length, h (0.06 meters) and an initial Concentration at time zero for each component, CO. Section 2 is the remaining 6 cm of the tubing and has an initial concentration at time zero, C. Section 1 of the tube is filled with compositions designated by 1 (e.g. C₁, D₁, E₁, F₁ and G₁) which comprise fragrance formulation C. Section 2 was filled with the corresponding composition designated by 2 (e.g. C₂, D₂, E₂, F₂ and G₂), these compositions do not comprise fragrance formulation C. Each tube was filled with coordinating compositions, for example composition C₁ was filled in Section 1 proximate to composition C₂ filled in Section 2 of the same tube. The tubes were sealed and aged for 60 days at 25° C. Following that time, samples were frozen, and three 2 cm sections were cut away from Section 2 and were analyzed individually for partitioned perfume component migration.

TABLE 9
Fragrance Formulation C

| | Molar | | |
|---|---|---|---|
| ClogP | Volume | Material Name | Amt |
| 1.05 | 112.2 | Heliotropin | 10.0 |
| 2.53 | 205.4 | Lyral | 12.0 |
| 2.2 | 156.2 | Eugenol | 2.5 |
| 3.81 | 196.1 | Ethyl Linalool | 15.0 |
| 4.24 | 219.8 | Ionone Beta | 25.0 |
| 3.72 | 203.6 | P.t.bucinal | 15.0 |
| 4.07 | 219.6 | Polysantol | 5.0 |
| 5.28 | 246.3 | Iso E Super | 15.0 |

Table 9 shows the fragrance components in Fragrance Formulation C which were used in Compositions C, D, E, F and G.

TABLE 10
% Migration of fragrance components from Compositions in Zone 1
to the 2 cm section in zone 2 most proximate to zone 1

| | | | | | | p.t. | | |
|---|---|---|---|---|---|---|---|---|
| | | Lyral | | ethyl | Beta | bucinal | | iso e super |
| | heliotropin | major | eugenol | linalool | ionone | (lilial) | polysantol | major |
| Formula | 3.4 | 4.6 | 0.0 | 0.0 | 0.4 | 0.0 | 0.0 | 0.0 |
| C.2 | | | | | | | | |
| Formula | 6.0 | 8.3 | 0.0 | 0.0 | 0.6 | 0.0 | 0.0 | 0.0 |
| D.2 | | | | | | | | |
| Formula | 3.6 | 19.2 | 9.7 | 7.1 | 2.4 | 2.2 | 0.0 | 1.8 |
| E.2 | | | | | | | | |
| Formula | 2.3 | 11.4 | 0.0 | 3.4 | 0.7 | 0.8 | 0.0 | 0.0 |
| F.2 | | | | | | | | |
| Formula | 8.1 | 9.7 | 0.0 | 3.8 | 1.7 | 1.4 | 0.0 | 0.0 |
| G.2 | | | | | | | | |

TABLE 11
% Migration of fragrance components from Compositions in
Zone 1 to the 2 cm section in zone 2 most proximate to zone 1

| | | | % of fragrance components in | | | | |
|---|---|---|---|---|---|---|---|
| | | | the compositions detected | | | | |
| | | | in the 2 cm section in zone 2 | | | | |
| | Molar | Material | most proximate to zone 1 | | | | |
| ClogP | Volume | Name | C₂, | D₂, | E₂, | F₂ | G₂ |
| 1.05 | 112.2 | Heliotropin | 3.4 | 6.0 | 3.6 | 2.3 | 8.1 |
| 2.53 | 205.4 | Lyral | 4.6 | 8.3 | 19.2 | 11.4 | 9.7 |
| 2.2 | 156.2 | Eugenol | 0.0 | 0.0 | 9.7 | 0.0 | 0.0 |
| 3.81 | 196.1 | Ethyl Linalool | 0.0 | 0.0 | 7.1 | 3.4 | 3.8 |
| 4.24 | 219.8 | Ionone Beta | 0.4 | 0.6 | 2.4 | 0.7 | 1.7 |
| 3.72 | 203.6 | P.t.bucinal | 0.0 | 0.0 | 2.2 | 0.8 | 1.4 |
| 4.07 | 219.6 | Polysantol | | | | | |
| 5.28 | 246.3 | Iso E Super | 0.0 | 0.0 | 1.8 | 0.0 | 0.0 |

Table 10 and Table 11 is the percent migration of fragrance components from the compositions from Zone 1 to the 2 cm section in zone 2 most proximate to Zone 1. This section of Zone 2 was directly proximate to the zone 1, zone 1 containing the composition which comprised fragrance composition C.

The unexpected result of this data was that percent migration is a function of C log P, molar volume, and zero-shear viscosity of the composition. Using the data along with the restricted diffusion analytical solution from Crank, Mathematics of Diffusion, 2^{nd} edition, pg. 63,

Restricted Diffusion Analytical Solution

f(x) = Co for 0 < x < h
f(x) = 0 for h < x < l
$\frac{C}{{C}_{0}} = \frac{h}{l} + \frac{2}{π} {\sum }_{n = 1}^{∞} \frac{1}{n} sin \frac{n π h}{l} exp ( - \frac{{Dn}^{2} {π}^{2} t}{{l}^{2}} ) cos \frac{n π x}{l}$
From Crank, Mathematics of Diffusion, 2^{nd} Ed. Pg. 63.

where Cₒ is the initial concentration contained within zone 1, C is the initial concentration contained within zone 2, l is the length (m) of the system, h is the length (m) of zone 1, and t is time (seconds)(As shown in FIG. 4)

a mathematical formula was derived by the inventor to predict the diffusion coefficient for a partitioned perfume component in a surfactant containing system. The equation is defined as:

D=(5.0481×10⁻⁶−(6.727×10⁻⁷*C log P)−(3.96×10⁻⁴/Molar Volume)+(3.254×10⁻⁴ /Zero-shear viscosity))²

Where C log P is unitless, molar volume is in cm³/mol and Zero-shear viscosity is the zero-shear viscosity (Pa·s) of the composition containing the partitioned component of interest.

Using this equation combined with the Restricted Diffusion Analytical Solution, the inventors have been able to model the ideal system that satisfies a stable system. In this case, a system is stable such that:

D≦5.3×10¹² m²/s

In one aspect of the present invention, the first personal care composition has a first fragrance character and the second personal care composition has a second fragrance character; wherein the first fragrance character is distinct from the second fragrance character, according to the Fragrance Differentiation Method.

While not being limited to the following, the inventors have included the following experimental examples to illustrate the property of perfume character.

Compositions H, I and J are prepared and comprise different fragrances, as shown in Table 12 and 13 below. Three compositions having three different fragrance formulations were prepared according to the formulas in Table 12 and Table 13. The compositions were prepared by conventional mixing techniques in the order of addition indicated. Addition step 8 in Table 12 containing Tridecyl Alcohol, PEG-90M, Xanthan Gum and Hydroxypropyl Guar was premixed prior to addition to the batch.

TABLE 12
Compositions H, I and J comprising different fragrances

| | H | I | J |
|---|---|---|---|
| 1. Water | QS | QS | QS |
| 2. Sodium Trideceth-3 Sulfate | 8.5 | 8.5 | 8.5 |
| 3. Sodium Lauryl Sulfate | 8.5 | 8.5 | 8.5 |
| 4. Sodium Lauroamphoacetate | 5.0 | 5.0 | 5.0 |
| 5. Disodium EDTA | 0.15 | 0.15 | 0.15 |
| 6. Sodium Benzoate | 0.2 | 0.2 | 0.2 |
| 7. Sodium Chloride | 4.7 | 4.7 | 4.7 |
| 8. PEG-90M | 0.15 | 0.15 | 0.15 |
| Xanthan Gum | 0.22 | 0.22 | 0.22 |
| Tridecyl Alcohol | 2 | 2 | 2 |
| Hydroxypropyl Guar | 0.6 | 0.6 | 0.6 |
| 9. Citric Acid | 0.84 | 0.84 | 0.84 |
| 10. Fragrance Formulation D | 1.0 | — | — |
| 10. Fragrance Formulation E | — | 1.0 | — |
| 10. Fragrance Formulation F | | | 1.0 |
| 11. Methyl Chloro Isothiazolinone & | 0.0005 | 0.0005 | 0.0005 |
| Methyl Isothiazolinone | | | |
| pH | 5.8 | 5.8 | 5.8 |

Three fragrance formulations as shown in Table 13 were formulated with different compositions, all having a tropical character.

TABLE 13
Fragrance Formulations D, E and F for use in Compositions H, I and J

| | Fragrance | Fragrance | Fragrance |
|---|---|---|---|
| Material Name | Formulation D | Formulation E | Formulation F |
| Minors 1% or less | QS | QS | QS |
| Benzyl Acetate | 2.094 | 2.326 | — |
| Benzyl Salicylate | 8.639 | 1.744 | — |
| Cis 3 Hexenyl Acetate | 1.047 | — | 2.000 |
| Cis-3-hexenyl Salicylate | 2.618 | 9.593 | 10.000 |
| Citronellol | 1.309 | — | — |
| Coumarine | 1.309 | 1.453 | — |
| Cyclemax | — | — | 3.000 |
| Cymal | — | — | 10.000 |
| Dimethyl Benzyl Carbinyl Butyrate | — | — | 5.000 |
| Ethylene Brassylate | 10.209 | 11.337 | — |
| Ethyl Linalool | 3.141 | 3.488 | — |
| Ethyl Methyl Phenyl Glycidate | — | — | 10.000 |
| Ethyl Trimethyl Cyclopentene | 1.963 | 2.18 | — |
| Butanol | | | |
| Ethyl-2-methyl Butyrate | — | — | 2.000 |
| Ethyl Vanillin | — | 1.453 | — |
| Florosa | 9.162 | 11.919 | — |
| Fructone | — | — | 3.000 |
| Geranyl Acetate | — | — | 4.000 |
| Hexyl Acetate | — | — | 3.000 |
| Italian Bergamont Oil Reggio | 1.571 | — | — |
| Ionone Beta | — | — | 8.000 |
| Ionone Gamma Methyl | 2.356 | 2.616 | — |
| Iso E Super | 4.188 | — | — |
| Jasmolactone | — | 1.163 | — |
| Linalool | — | — | 10.000 |
| Methyl Dihydro Jasmonate | 29.319 | 32.558 | — |
| Methyl Phenyl Carbinyl Acetate | — | — | 2.000 |
| Nonalactone | 3.272 | 2.326 | — |
| Oil Lime Expressed Mex Fcc 150421 | — | 1.453 | — |
| Para Hydroxy Phenyl Butanone | — | — | 3.000 |
| Phenyl Ethyl Alcohol | 3.927 | — | — |
| Prenyl Acetate | — | — | 2.000 |
| P.T. Bucinal | 6.675 | 7.413 | — |
| Undecalactone | — | — | 2.000 |
| Undecavertol | — | — | 3.000 |
| Verdox | — | — | 10.000 |

The compositions of Table 12 were prepared. Fifteen non-expert panelists were asked to assess these fragrances. The fifteen panelists were asked to determine if one composition comprising one fragrance formulation smelled different from another composition comprising another fragrance formulation, according to the fragrance differentiation method. 15 non-expert graders were asked to smell three containers according to the fragrance differentiation method, the results were as follows:

Test 1: One container contained composition H containing fragrance formulation D and two containers contained composition I containing fragrance formulation E. 8 of the 15 individuals identified the correct container that had a different odor than the other two containers. This is not a statistically significant difference, therefore characters of the two perfume formulations not considered to be distinct according to the Fragrance Differentiation Method.

Test 2: Two containers contained composition I containing fragrance formulation E and one container contained composition J containing fragrance formulation F. 13 of the 15 individuals identified the correct container that had a different odor than the other two containers. This is a statistically significant result, therefore the characters of the two perfume formulations are considered to be distinct according to the Fragrance Differentiation Method.

Different fragrances can be selected from the following fragrance non-limiting character groups: citrus, green, floral, fruity, berry, tropical, melon, aldehydic floral, spicy, woody, oriental, chypre, musk, citrus, fougere, herbaceous, fresh, woody, leathery, ambery, aromatic, watery, balsamic, floriental and mixtures thereof.

Fragrance pairs can be chosen from the same fragrance groups, for example the composition contained in Zone 1 contains a fragrance from the fruity group that has an apple character and the composition contained in Zone 2 contains a fragrance from the fruity group that has a pear character.

Likewise, fragrance pairs can be chosen from different fragrance groups, for example the composition contained in Zone 1 contains a fragrance from the citrus group that has an orange character and the composition contained in Zone 2 contains a fragrance from the green group that has a pine character.

Preferred fragrance pairs include, but are not limited to: lavender and vanilla; lavender and aloe; aloe and vanilla; berry and vanilla; pear and aloe; orange and grapefruit; coconut and berry; melon and cucumber; tropical and floral; citrus and green; fresh and citrus; oriental and fruity; herbaceous and green; floral and fruity; spicy and woody; floriental and floral; watery and fresh.

In some aspects of the present invention, each personal care composition disposed with each zone may have a fragrance with a distinct character, so that every personal care composition within the personal care product is distinct from the next. For example, the personal care product can begin with a personal care composition comprising a fragrance with an orange character, the next personal care composition can comprise a lemon character and a third personal care composition can comprise a grapefruit character. In other aspects, the personal care composition disposed with in a zone may comprise a fragrance with a first character which can alternate with another personal care composition with a second character throughout the product. For example, the personal care product can begin with a personal care composition comprising a fragrance with a vanilla character, followed by a personal care composition comprising a fragrance with a lavender character, followed by a personal care composition comprising a fragrance with a vanilla character and a final personal care composition comprising a fragrance with a lavender character.

The compositions of the present invention can be multi-phase and comprise one of more phases or one or more of the components described in the phases below:

The personal care composition of the present invention can comprise a cleansing phase or cleansing phase components. The personal care composition typically comprises from about 1% to about 100%, by weight of the composition; from about 5% to about 85%; by weight of the composition, from about 10% to 80%, by weight of the composition; from about 20 to 70%, by weight of the composition; from about 25% to 60%, by weight of the composition, from about 30% to about 50%, by weight of the composition, of a cleansing phase.

The cleansing phase can comprise a structured domain that is comprised of a mixture of surfactants. The presence of structured domain enables the incorporation of high levels of benefit components in a separate phase which is not emulsified within composition. In one aspect, the structured domain in the composition can be characterized as, or is, an opaque structured domain. In one aspect, the opaque structured domain can be characterized as, or is, a lamellar phase. The lamellar phase produces a lamellar gel network. The lamellar phase can provide resistance to shear, adequate yield to suspend particles and droplets and at the same time provides long term stability, since it is thermodynamically stable. The lamellar phase tends to have a higher viscosity thus minimizing the need for viscosity modifiers.

In one aspect, cleansing phase can comprise a domain that is comprised of a mixture of surfactants and can be a micellar phase. A micellar phase is optically isotropic. Micelles are approximately spherical in shape. Other shapes such as ellipsoids, cylinders, and bilayers are also possible. In one aspect, the micellar phase can be structured to enhance viscosity and to suspend particles. This can be accomplished using viscosity modifiers such as those defined below as water structurants.

The cleansing phase comprises a surfactant component which can be comprised of a mixture of surfactants including lathering surfactants or a mixture of lathering surfactants. The cleansing phase comprises surfactants suitable for application to the mammalian skin or hair and are compatible with water and the other ingredients of the composition of the present invention. These surfactants include anionic, nonionic, cationic, zwitterionic, amphoteric, soap, or combinations thereof. Preferably, anionic surfactant comprises at least 40% of the surfactant component. The personal care composition can comprise the surfactant component at concentrations ranging from about 2% to about 40%, from about 4% to about 25%, about 1% to about 21%, about 3% to about 15%, by weight of the composition, of the surfactant component.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975.

Preferred linear anionic surfactants for use in the structured surfactant phase of the personal care composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anionic surfactants and monomethyl branched anionic surfactants suitable for the present invention are described in a commonly owned, patent application published on December, 2006 under U.S. Publication No. 60/680,149 entitled “Structured Multi-phased Personal Cleansing Compositions Comprising Branched Anionic Surfactants” filed on May 12, 2005 by Smith, et al. Branched anionic surfactants include but are not limited to the following surfactants: sodium trideceth sulfate, sodium tridecyl sulfate, sodium C₁₂₋₁₃ alkyl sulfate, and C₁₂₋₁₃ pareth sulfate and sodium C₁₂₋₁₃ pareth-n sulfate.

In one aspect of the personal care compositions of the present invention may further preferably comprise an amphoteric surfactant, a zwitterionic surfactant and mixtures thereof. In one embodiment, the personal care composition can comprise at least one amphoteric surfactant. Amphoteric surfactant suitable for use in the present invention include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In one aspect, the personal care composition can comprise an amphoteric surfactant that is selected from the group consisting of sodium lauroamphoacetate, sodium cocoamphoactetate, disodium lauroamphoacetate disodium cocodiamphoacetate, and mixtures thereof. Moreover, Amphoacetates and diamphoacetates can also be used.

Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the personal care composition include alkyl betaines, including cocoamidopropyl betaine.

The personal care composition of the present invention is preferably free of alkyl amines and alkanolamide to ensure mildness of the composition to the skin.

An electrolyte can be added per se to the personal care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium chloride, ammonium chloride, sodium or ammonium sulfate. The electrolyte is preferably added to the structured surfactant phase of the composition in the amount of from about 0.1% to about 6%; from about 1% to about 5%, more preferably from about 2% to about 4%, more preferably from about 3% to about 4%, by weight of the personal care composition.

The first personal care composition can comprise a first concentration of surfactant and second personal care composition can comprise a second concentration of surfactant. The first concentration of surfactant can be different from the second concentration of surfactant. In one aspect, the first personal care composition can a first concentration of surfactant that is a greater that the second concentration of surfactant in the second personal care compositions. In one aspect, the first personal care composition can have a lower concentration of surfactant than the second personal care compositions.

The personal care compositions of the present invention comprise a benefit phase or benefit phase components. The benefit phase in the present invention is preferably anhydrous and can be substantially free of water. The benefit phase can be substantially free or free of surfactant. The benefit phase typically comprises hydrophobic benefit materials. The benefit phase may comprise from about 1% to about 50%, preferably from about 5% to about 30%, more preferably from about 10% to about 30%, by weight of the personal care composition, of a hydrophobic benefit material.

Hydrophobic benefit materials suitable for use in the present invention preferably have a Vaughan Solubility Parameter of from about 5 (cal/cm³)^{1/2} to about 15 (cal/cm³)^{1/2}, as defined by Vaughan in Cosmetics and Toiletries, Vol. 103. The Vaughan Solubility Parameter (VSP) as used herein is a parameter used to define the solubility of hydrophobic materials. Vaughan Solubility parameters are well known in the various chemical and formulation arts and typically have a range of from 5 to 25. Non-limiting examples of hydrophobic benefit materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility, Effects in Product, Package, Penetration and Preservation, C. D. Vaughan, Cosmetics and Toiletries, Vol. 103, October 1988.

The hydrophobic benefit materials for use in the benefit phase of the composition have a preferred rheology profile as defined by Consistency value (k) and Shear Index (n). The term “Consistency value” or “k” as used herein is a measure of lipid viscosity and is used in combination with Shear Index, to define viscosity for materials whose viscosity is a function of shear. The measurements are made at 35° C. and the units are poise (equal to 100 cps). The term “Shear Index” or “n” as used herein is a measure of lipid viscosity and is used in combination with Consistency value, to define viscosity for materials whose viscosity is a function of shear. The measurements are made at 35° C. and the units are dimensionless. Consistency value (k) and Shear Index (n) are more fully described in the Test Methods below. Preferred Consistency value ranges are 1-10,000 poise (1/sec)ⁿ⁻¹, preferably 10-2000 poise (1/sec)ⁿ⁻¹ and more preferably 50-1000 poise (1/sec)ⁿ⁻¹. Shear Index ranges are 0.1-0.8, preferably 0.1-0.5 and more preferably 0.20-0.4. These preferred rheological properties are especially useful in providing the personal cleansing compositions with improved deposition of benefit agents on skin.

The benefit phase can be comprised of the hydrophobic benefit materials selected from the group consisting of petrolatum, lanolin, derivatives of lanolin (e.g. lanolin oil, isopropyl lanolate, acetylated lanolin, acetylated lanolin alcohols, lanolin alcohol linoleate, lanolin alcohol riconoleate) hydrocarbon oils (e.g. mineral oil) natural and synthetic waxes (e.g. micro-crystalline waxes, paraffins, ozokerite, lanolin wax, lanolin alcohols, lanolin fatty acids, polyethylene, polybutene, polydecene, pentahydrosqualene) volatile or non-volatile organosiloxanes and their derivatives (e.g. dimethicones, cyclomethicones, alkyl siloxanes, polymethylsiloxanes, methylphenylpolysiloxanes), natural and synthetic triglycerides (e.g. castor oil, soy bean oil, sunflower seed oil, maleated soy bean oil, safflower oil, cotton seed oil, corn oil, walnut oil, peanut oil, olive oil, cod liver oil, almond oil, avocado oil, palm oil, sesame oil) and combinations thereof. The benefit phase of the personal care composition can be comprised a combination of petrolatum and mineral oil.

The personal care compositions of the present invention can comprise a structured aqueous phase which can comprise a water structurant and water. The structured aqueous phase can be hydrophilic and in one aspect, can be a hydrophilic, non-lathering gelled water phase. The structured aqueous phase can comprises less than about 5%; less than about 3%; less than about 1%, by weight of the structured aqueous phase, of a surfactant component and, in one aspect, can be free of lathering surfactants. The structured aqueous phase of the present invention can comprise from about 30% to about 99%, more than about 50%, more than about 60%, more than about 70%, more than about 80%, by weight of the structured aqueous phase, of water.

The water structurant is selected from the group consisting of inorganic water structurants (e.g. silicas, polyacrylates, polyacrylamides, modified starches, crosslinked polymeric gellants, copolymers) charged polymeric water structurants (e.g. Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), water soluble polymeric structurants (e.g. cellulose gums and gel, and starches), associative water structurants (e.g. xanthum gum, gellum gum, pectins, alginates such as propylene glycol alginate), and mixtures thereof. The structured aqueous phase can comprise from about 0.1% to about 30%, from about 0.5% to about 20%, from about 0.5% to about 10%, and from about 0.5% to about 5%, by weight of the structured aqueous phase, of a water structurant. The water structurant for the structured aqueous phase can have a net cationic charge, net anionic charge, or neutral charge. The structured aqueous phase can have a pH in the range from about 5 to about 9.5, or in one aspect have a pH of about 7.

While not essential for the purposes of the present invention, the non-limiting list of optional materials, illustrated hereinafter are suitable for use in personal care compositions, and may be incorporated in certain embodiments, for example to assist or enhance cleansing performance, for treatment of the skin, or to modify the aesthetics of the personal care composition. Optional materials useful in the products herein are described by their cosmetic and/or therapeutic benefit or their postulated mode of action or function. These descriptions are non-limiting and made for the sake of convenience because it is understood that these materials can provide more than one benefit, function or operate via more than one mode of action. The precise nature of these optional materials, and levels of incorporation thereof, will depend on the physical form of the composition and the nature of the cleansing operation for which it is to be used. The amount of optional materials in compositions are usually formulated, by weight of the composition, at less than about less than about 6%, less than about 5%, less than about 4%, less than about 3%, less than about 2%, less than about 1%, less than about 0.5%, less than about 0.25%, less than about 0.1%, less than about 0.01%, less than about 0.005%.

Optional ingredients, which can be used in the personal care compositions of the present invention, can be selected from the group consisting of thickening agents; low density microspheres (e.g. Expancel 091 WE40 d24, Akzo Nobel and others described in commonly owned and assigned U.S. Patent Publication No. 2004/0092415A1 published on May 13, 2004); preservatives; antimicrobials; fragrances; chelators (e.g. such as those described in U.S. Pat. No. 5,487,884 issued to Bisset, et al.); sequestrants; vitamins (e.g. Retinol); vitamin derivatives (e.g. tocophenyl actetate, niacinamide, panthenol); sunscreens; desquamation actives (e.g. such as those described in U.S. Pat. No. 5,681,852 and 5,652,228 issued to Bisset); anti-wrinkle/ anti-atrophy actives (e.g. N-acetyl derivatives, thiols, hydroxyl acids, phenol); anti-oxidants (e.g. ascorbic acid derivatives, tocophenol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); polymeric phase structurant (e.g. naturally derived polymers, synthetic polymers, crosslinked polymers, block copolymers, copolymers, hydrophilic polymers, nonionic polymers, anionic polymers, hydrophobic polymers, hydrophobically modified polymers, associative polymers, and oligomers); a liquid crystalline phase inducing structurant (e.g. trihydroxystearin available from Rheox, Inc. under the trade name THIXCIN® R); organic cationic deposition polymer (e.g. Polyquaternium 10 available from Amerchol Corp. Edison, N.J., USA, guar hydroxypropyltrimonium chloride available as Jaguar C-17 from Rhodia Inc., and N-Hance polymer series commercially available from Aqualon); pH regulators (e.g. triethanolamine); anti-acne medicaments; essential oils; sensates; pigments; colorants; pearlescent agents; interference pigments (e.g such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A by Taylor, et al.) and mixtures thereof. Other optional ingredients are most typically those materials approved for use in cosmetics and that are described in the CTFA Cosmetic Ingredient Handbook, Second Edition, The Cosmetic, Toiletries, and Fragrance Association, Inc. 1988, 1992.

### Test Methods

### Dialysis Method:

The Dialysis Method is for determining the migration, or diffusion over time, of chemical partitioned components from one composition of a dual-composition system to a second composition of a dual-composition system. It is designed for viscous materials. Migration is accelerated using a cell with two chambers divided by a dialysis cell, as described below. The bulk of the compositions are kept separate but molecules smaller than 3,500 MW are free to diffuse. The high surface area to thickness ratio allows diffusion to go to equilibrium in a manageable time frame. The materials needed are: a dialysis cell (described below), a dialysis membrane composed of regenerated cellulose with a molecular weight cut off of 3,500, available from Pierce Biotechnology of Thermo Fisher Scientific (Pierce Biotechnology, Inc.; P.O. Box 117; Rockford, Ill. 61105 product no. 68035) which is cut open to lay flat; clamps; disposable syringes; and a flat-edged spatula

In the case of testing from a product package, two zones can be selected from the package that contains at least two compositions that contain the separate fragrance formulations. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions.

Loading and Unloading compositoins into dialysis cell: A first endplate made of Plexiglas™ having the dimensions of 6 inches in length, 5 inches in width and ½ inch depth is placed on a flat surface and topped with first gasket made of silicone rubber having same dimensions as end plate, with a cutout in the center that has the dimension of 4 inches in length by 1½ inches in width. The gasket is pressed down to form a seal with the endplate, then 20 grams of the first composition in a disposable syringe is dispensed into the space in the gasket. The dialysis membrane, having similar in dimensions to endplate and the first gasket, is placed on top of this and pressed down to form a seal with the first gasket. A second gasket made of the same material and same dimensions as the first gasket is placed on top of the dialysis membrane and pressed down. The second compostion is then dispensed into the space in the second gasket on top of the dialysis membrane. This is topped with the second endplate, having dimensions and made similar in materials as the first endplate, and the entire assembly is held together with clamps. It can be placed vertically on a flat surface for the duration of 1 week at 25° C. To remove the test materials, place the diffusion cell flat and disassemble in the reverse order, scraping each material out with a flat-edged spatula as it is exposed. Each composition is analyzed individually for partitioned components according to the Gas Chromatograph Method described hereinafter.

### Gas Chromatograph Method:

The Internal Standard Solution used is a mixture of 150 mg diphenyl oxide dissolved in 500 ml methanol. The Calibration Solution is made by adding 10 mg neat perfume to a vial that contains 5 ml of the internal standard solution and 15 ml methanol.

The samples were prepared, as follows: First, weigh between 1 and 5 g of a composition into a vial, add 5 ml internal standard solution and 15 ml methanol. Next, shake the vial vigorously to disperse. Next, filter the contents of the vial through a Acrodisc syringe filter (PVDF, 25 mm diameter, 0.45 um pore size). If one is unable to filter through the PVDF, 25 mm diameter 0.45 um pore size filter, sample solutions may be pre-filtered through Glass Fiber Acrodisc syringe filter (37 mm, 1 um pore size).

The instrument parameters of the gas chromatograph and the mass spectrometer are shown in the chart below:

| | |
|---|---|
| Gas Chromatograph | |
| Column | DB-1, 60 M × 0.32 mm I.D., 1.0 um film thickness |
| | (J&W cat. No. 123-1063) |
| Column Flow | 1.2 ml helium/min, constant flow mode |
| Injector Temp | 250° C. |
| Injection Mode | 1 ul, split @ 20:1 |
| Oven Program | Initial temp 50° C., initial time 2.0 min, ramp |
| | 4° C./min, final temp 280° C., final time 5.0 min. |
| Mass Spectrometer | |
| Transfer Line Temp | 280° C. |
| Solvent Delay | 4.0 min. |
| Scan Parameters | Low mass 35.0 |
| | High mass 350.0 |
| | Threshold 150 |
| Quad Temp | 150° C. |
| Source Temp | 230° C. |

Operation: The calibration solution is injected. The peaks of interest are identified and the instrument is calibrated. The sample solutions are injected sample solutions and calibrated peaks are quantified.

### Fragrance Differentiation Method:

For the present invention, change in fragrance is defined such that the composition containing a fragrance formulation contained within the first zone smells distinct from a composition containing a fragrance formulation contained within the second zone. A distinct fragrance can be quickly assessed by the following procedure.

In the case of testing from a product package, two zones can be selected from the package that contains at least two compositions that contain the separate fragrance formulations. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions.

Three coded compositions contained in a 4 oz glass jar with a cap are placed before non-expert panelists, in which two of the glass jars contain 10 g of a composition containing a first perfume formulation and one of the glass jars contains 10 g of a composition containing a second perfume formulation; starting from the left, each glass jar is evaluated the and the panelist is asked to identify the composition containing a second fragrance formulation that is distinct from the other two compositions that they think contains the first fragrance formulation. The compositions containing the fragrance formulations in the glass jar may be reevaluated but the panelist must identify what they believe is the composition containing the second perfume formulation that smells distinct from the other two compositions that they think contains the first fragrance formulations. The order is randomized for each panelist.

The number of panelists needed to correctly identify the distinct sample is defined in Milton, Introduction to Probability and Statistics, 4^{th} edition, pg. 317. (Section 9.2: Testing Hypotheses on a Proportion). Significance is calculated at 95% confidence.

### T-Bar Viscosity Method:

The viscosity of a composition contained in a zone can be assessed by the T-Bar Viscosity Method. In the case of testing from a product package, two zones can be selected from the package that contains at least two compositions that contain the separate fragrance formulations. In order to separate said zones, the product can be frozen at a temperature of at least −20° C. for a period of at least 24 hours. Said zones are then cut using a cutting implement such as a bandsaw. The cut portions are collected separately and allowed equilibrate to ambient conditions. The apparatus for T-Bar measurement includes a Brookfield DV-II+ Pro Viscometer with Helipath Accessory; chuck, weight and closer assembly for T-bar attachment; a T-bar Spindle D, a personal computer with Rheocalc software from Brookfield, and a cable connecting the Brookfield Viscometer to the computer. First, weigh 80 grams of the first or second composition in a 4-oz glass jar. Measure the T-bar viscosity by carefully dropping the T-Bar Spindle to the interior bottom of the jar and set the Helipath stand to travel in an upward direction. Open the Rheocalc software and set the following data acquisition parameters: set Speed to 5 rpm, set Time Wait for Torque to 00:01 (1 second), set Loop Start Count at 100. Start data acquisition and turn on the Helipath stand to travel upward at a speed of 22 mm/min. The T-Bar viscosity “T,” is the average T-Bar viscosity reading between the 6^{th} reading and the 95^{th} reading (the first five and the last five readings are not used for the average T-Bar viscosity calculation). If the viscosity is below the lower limit of the D spindle (30,000 cps), a larger spindle can be used for the T-Bar Viscosity measurement.

### Ultracentrifugation Method:

The Ultracentrifugation Method is used to determine the percent of a structured domain or an opaque structured domain that is present in a multi-phase personal care composition that comprises a structured surfactant phase comprising a surfactant component. The method involves the separation of the composition by ultracentrifugation into separate but distinguishable layers. The multi-phase personal care composition of the present invention can have multiple distinguishable layers, for example a non-structured surfactant layer, a structured surfactant layer, and a benefit layer.

First, dispense about 4 grams of multi-phase personal care composition into Beckman Centrifuge Tube (11×60 mm). Next, place the centrifuge tubes in an Ultracentrifuge (Beckman Model L8-M or equivalent) and ultracentrifuge using the following conditions: 50,000 rpm, 18 hours, and 25° C.

After ultracentrifuging for 18 hours, determine the relative phase volume by measuring the height of each layer visually using an Electronic Digital Caliper (within 0.01 mm). First, the total height is measured as Hₐ which includes all materials in the ultracentrifuge tube. Second, the height of the benefit layer is measured as H_{b}. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:

Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacings of the liquid crystal structure. There may be several structured layers present, so that H_{c} is the sum of the individual structured layers. If a coacervate phase or any type of polymer-surfactant phase is present, it is considered a structured phase.

Finally, the structured domain volume ratio is calculated as follows:

Structured Domain Volume Ratio=H_{c}/Hₛ*100%

If there is no benefit phase present, use the total height as the surfactant layer height, Hₛ=Hₐ.

### Yield Stress and Zero Shear Viscosity Method:

The Yield Stress and Zero Shear viscosity of a composition contained in a zone can be assessed by the Yield Stress and Zero Shear Viscosity method. In the case of testing from a product package, two zones can be selected from the package that contains at least two compositions that contain the separate fragrance formulations. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress. Serrated plates can be used to obtain consistent results when slip occurs.

First a sample of the composition is obtained and placed in position on the rheometer base plate, the measurement geometry (upper plate) moving into position 1 mm above the base plate. Excess composition at the geometry edge is removed by scraping after locking the geometry. If the composition comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a composition has any particle larger than 5 mm in any dimension, the particles are removed prior to the measurement.

The determination is performed via the programmed application of a continuous shear stress ramp from 0.1 Pa to 1,000 Pa over a time interval of 4 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:

Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., lox for each x). The predicted strain is compared to the actual strain at each measurement point to obtain a % variation at each point, using Equation (2).

% variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

### EXAMPLES

### Example 1

A personal care article was prepared which contains two compositions with different fragrance characters according to the fragrance differentiation method. The two compositions used have two different fragrance formulations were prepared according to the formulas in Table 14 and the fragrance compositions in table 13, shown above. The compositions were prepared by conventional mixing techniques in the order of addition indicated. Addition step 8 in Table 14 containing Tridecyl Alcohol, PEG-90M, Xanthan Gum and Hydroxypropyl Guar was premixed prior to addition to the batch.

TABLE 14
Compositions K and L comprising different fragrances

| | K | L |
|---|---|---|
| 1. Water | QS | QS |
| 2. Sodium Trideceth-3 Sulfate | 6.74 | 6.74 |
| 3. Sodium Lauryl Sulfate | 6.73 | 6.73 |
| 4. Sodium Lauroamphoacetate | 3.99 | 3.99 |
| 5. Disodium EDTA | 0.12 | 0.12 |
| 6. Sodium Benzoate | 0.16 | 0.16 |
| 7. Sodium Chloride | 4.00 | 4.00 |
| 8. PEG-90M | 0.12 | 0.12 |
| Xanthan Gum | 0.17 | 0.17 |
| Tridecyl Alcohol | 1.58 | 1.58 |
| Hydroxypropyl Guar | 0.48 | 0.48 |
| 9. Citric Acid | 0.75 | 0.75 |
| 10. Fragrance Formulation E | 1.0 | — |
| 10. Fragrance Formulation F | — | 0.7 |
| 11. Methyl Chloro Isothiazolinone & Methyl | 0.0004 | 0.0004 |
| Isothiazolinone | | |
| pH | 5.5 | 5.5 |

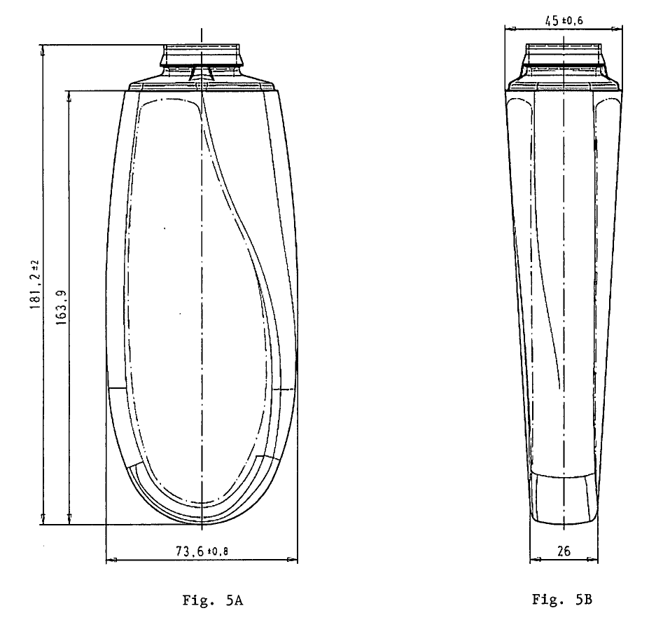

The compositions are tilled in a tottle with a volume of 290 ml as specified in FIGS. 5A and 5B. Zone 1 included the upper 30% (87 ml) of the tottle volume, which is filled with composition K, which has a character of coconut. Zone 2 included the middle 40% (116 ml) of the tottle volume, which is filled with composition L, which has a character of berry. Zone 3 included the lower 30% (87 ml) of the tottle, which is filled with composition K, which has a character of coconut. The tottle was dispensed at 10 ml per dose, the fragrance character changes from coconut (first 9 days of use) to berry (second 11 days of use) to coconut (final 9 days of usage).

### Example 2

A personal care article was prepared which contains three compositions with three different fragrance characters according to the fragrance differentiation method. Three compositions having three different fragrance formulations were prepared according to the formulas in Table 15 and the fragrance compositions in Table 9 and Table 13, shown above. The compositions were prepared by conventional mixing techniques in the order of addition indicated. Addition step 6 in Table 15 containing water and Polyquaternium-10 was premixed prior to addition to the batch.

TABLE 15
Compositions M, N and O comprising different fragrances

| | M | N | O |
|---|---|---|---|
| 1. Sodium Lauryl Sulfate | 9.5 | 9.5 | 9.5 |
| 2. Sodium Laureth Sulfate | 5.7 | 5.7 | 5.7 |
| Adjust to pH 5 with Citric Acid | | | |
| 3. Acrylates Copolymer (Aqua SF-1) | 1.9 | 1.9 | 1.9 |
| 4. Coco Monoethanolamide | 0.76 | 0.76 | 0.76 |
| 5. Caustic Soda 50% | 0.16 | 0.16 | 0.16 |
| 6. Water | QS | QS | QS |
| Polyquaternium-10 | 0.24 | 0.24 | 0.24 |
| 7. Disodium EDTA | 0.12 | 0.12 | 0.12 |
| 8. Sodium Benzoate | 0.24 | 0.24 | 0.24 |
| 9. Citric Acid | 0.65 | 0.65 | 0.65 |
| 10. Fragrance Formulation C | 1.0 | — | — |
| 10. Fragrance Formulation E | — | 1.0 | — |
| 10. Fragrance Formulation F | — | — | 0.7 |
| 11. Methyl Chloro Isothiazolinone & Methyl | 0.0005 | 0.0005 | 0.0005 |
| Isothiazolinone | | | |
| pH | 5.4 | 5.4 | 5.4 |

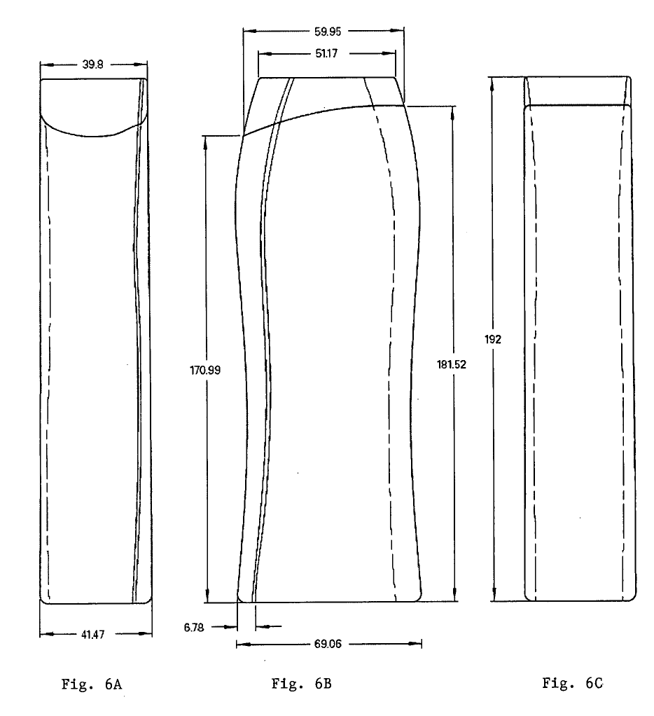

The compositions are filled in a bottle with a volume of 360 ml as specified in FIGS. 6A, 6B and 6C. Zone 1 included the upper 30% (111 ml) of the bottle volume, which is filled with composition M, which has a character of floral. Zone 2 included the middle 40% (148 ml) of the bottle volume, which is filled with composition N, which has a character of coconut. Zone 3 included the lower 30% (111 ml) of the bottle, which is filled with composition O, which has a character of berry. When the bottle is dispensed at 10 ml per dose, the fragrance character changes from floral (first 11 days of use) to coconut (second 14 days of use) to berry (final 11 days of usage).

### Example 3

A personal care article was prepared which contains two compositions with two different fragrance characters according to the fragrance differentiation method. Two compositions having two different fragrance formulations were prepared according to the formulas in Table 16. The compositions were prepared by conventional mixing techniques in the order of addition indicated. Addition step 8 in Table 16 containing Tridecyl Alcohol, PEG-90M, Xanthan Gum and Hydroxypropyl Guar was premixed prior to addition to the batch.

TABLE 16
Compositions P and Q comprising different fragrances

| | P | Q |
|---|---|---|
| 1. Water | QS | QS |
| 2. Sodium Trideceth-3 Sulfate | 6.74 | 6.74 |
| 3. Sodium Lauryl Sulfate | 6.73 | 6.73 |
| 4. Sodium Lauroamphoacetate | 3.99 | 3.99 |
| 5. Disodium EDTA | 0.13 | 0.13 |
| 6. Sodium Benzoate | 0.17 | 0.17 |
| 7. Sodium Chloride | 4.00 | 4.00 |
| 8. PEG-90M | 0.13 | 0.13 |
| Xanthan Gum | 0.19 | 0.19 |
| Tridecyl Alcohol | 1.68 | 1.68 |
| Hydroxypropyl Guar | 0.51 | 0.51 |
| 9. Citric Acid | 0.8 | 0.8 |
| 10. Lavender Fragrance | 1.0 | — |
| 10. Vanilla Fragrance | — | 1.0 |
| 11. D&C Violet #2 | 0.003 | — |
| 12. Methyl Chloro Isothiazolinone & Methyl | 0.0004 | 0.0004 |
| Isothiazolinone | | |
| pH | 5.5 | 5.5 |

The compositions are filled in a tottle with a usable volume of 290 ml and dimensions as specified in FIGS. 5A and 5B. Zone 1 includes the upper 50% (145 ml) of the tottle volume, which is filled with composition P, which has a character of Lavender. Zone 2 includes the lower 50% (145 ml) of the tottle volume, which is filled with composition Q, which has a character of Vanilla. When the tottle is dispensed at 10 ml per dose, the fragrance character changes from Vanilla (first 14 days usage) to lavender (second 14 days usage).

Control compositions are filled in a tottle with a usable volume of 290 ml and dimensions as specified in FIGS. 5A and 5B. One control is filled with 290 ml composition P, which has a character of Lavender. A second control is filled with 290 ml composition Q, which has a character of Vanilla.

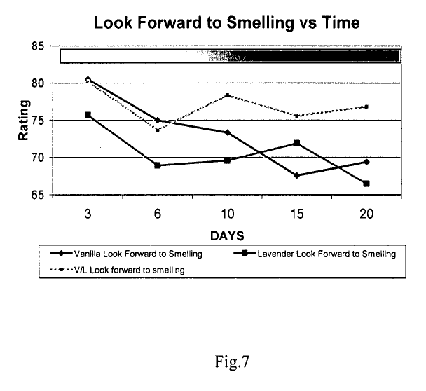

Consumers were then asked to evaluate the product over a usage period of 28 days or completion of the bottle. Questionnaires were used during the 28 day period to evaluate the consumer interest in their product. This data shown in FIG. 7 was graphed to monitor consumer anticipation of a change in fragrance during the usage of the personal care article of the 290 ml over the 28 days.

As shown in FIG. 7, during the period where the consumer experiences a shift from composition Q to composition P, consumers continue to look forward to experiencing the scent versus the single scented product. This interest continues during the time the product is transitioning and remains directionally higher than the single scented products.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A personal care article for dispensing and or applying a liquid personal care product comprising:
a) a single chamber package comprising a dispensing orifice, a first zone proximate to said dispensing orifice and a second zone distal to said dispensing orifice; and
b) a liquid personal care product comprising a first personal care composition substantially disposed in said first zone and a second personal care composition substantially disposed in said second zone;
wherein said first composition comprises a first fragrance character;
wherein said second composition comprises a second fragrance character; and
wherein said first fragrance character is distinct from said second fragrance character.

2) The personal care article of claim 1 wherein said first zone comprises from about 10% to about 90%, by volume, of said package.

3) The personal care article of claim 1 wherein said first zone comprises from about 30% to about 70%, by volume of said package.

4) The personal care article of claim 1 wherein said first zone comprises about 50%, by volume of said package.

5) The personal care article of claim 1, wherein said first personal care composition comprises a first concentration of surfactant and said second personal care composition comprises a second concentration of surfactant.

6) The personal care article of claim 1, wherein said first concentration of surfactant is greater than said second concentration of surfactant.

7) The personal care article of claim 1, wherein said first personal care composition comprises a first partitioned perfume component.

8) The personal care article of claim 7, wherein said first partitioned perfume component has a C log P of at least 2.

9) The personal care article of claim 7, wherein said first partitioned perfume component has a molar volume of from about 100 Angstrom to about 300 cm³.

10) The personal care article of claim 7, wherein said first partitioned perfume component has a diffusion coefficient of less than 5.3×10⁻¹² according to the restricted diffusion analytical solution.

11) The personal care article of claim 7, wherein said first partitioned perfume has a C log P, Molar Volume, and the system has a Zero-shear viscosity such that: (5.0481×10⁻⁶−(6.727×10⁻⁷* C log P)−(3.96×10⁻⁴/Molar Volume)+(3.254×10⁻⁴/Zero-shear viscosity))²≦5.3×10⁻¹²

12) The personal care article of claim 1, wherein said second personal care composition comprises a second partitioned perfume component.

13) The personal care article of claim 12, wherein said second partitioned perfume component has a C log P of at least 2.

14) The personal care article of claim 12, wherein said second partitioned perfume component has a molar volume of from about 100 Angstrom to about 300 cm³.

15) The personal care article of claim 12, wherein said second partitioned perfume component has a diffusion coefficient less than 5.3×10⁻¹² according to the restricted diffusion analytical solution.

16) The personal care article of claim 12, wherein said first partitioned perfume has a C log P, Molar Volume, and the system has a Zero-shear viscosity such that: (5.0481×10⁻⁶−(6.727×10⁻⁷*C log P)−(3.96×10⁻⁴/Molar Volume)+(3.254×10⁻⁴/Zero-shear viscosity))²≦5.3×10⁻¹²

17) The personal care article of claim 1, wherein said first personal care composition comprises a micellar phase.

18) The personal care article of claim 1, wherein said second personal care composition comprises a micellar phase.

19) The personal care article of claim 1, wherein said first personal care composition comprises a lamellar phase.

20) The personal care article of claim 1, wherein said second personal care composition comprises a lamellar phase.

21) The personal care article of claim 1 wherein said first personal care composition has a zero-shear viscosity is at least 500 Pascal.

22) The personal care article of claim 1 wherein said second personal care composition has a zero-shear viscosity is at least 500 Pascal.

23) The personal care article of claim 1, wherein said first personal care composition is substantially dispensed prior to dispensing said second personal care composition.

24) The personal care article of claim 1 wherein said first personal care composition is a distinct hue from said second personal care composition.

25) The personal care article of claim 1, wherein said first zone is in physical contact with said second zone within said package.

26) The personal care article of claim 1, further comprises a third zone which is medial to the dispensing orifice.

27) The personal care article of claim 26 further comprising a third personal care composition substantially disposed within said third zone; said third personal care composition comprising a third fragrance character wherein said third is distinct from said first fragrance character and said second fragrance character.
